(19) United States
(12) Patent Application Publication
(10) Pub. No.: US 2023/0317281 A1
(43) Pub. Date: Oct. 5, 2023

(54) TARGETED MEDICAL INTERVENTION SYSTEM

(71) Applicant: Express Scripts Strategic Development, Inc., St. Louis, MO (US)

(72) Inventors: Kristin A. Biedinger, Pittsburgh, PA (US); Randy L. Canis, Chesterfield, MO (US)

(21) Appl. No.: 17/712,346

(22) Filed: Apr. 4, 2022

(51) Int. Cl. G16H 50/20; G16H 10/60; G16H 40/67

(52) U.S. Cl. CPC G16H 50/20; G16H 10/60; G16H 40/67

(57) ABSTRACT

Targeted medical intervention system comprises a processor that performs operations comprising selecting target user and generating targeted subset from population of users. To generate the targeted subset, the processor, compares each of the medical claim histories of the population of users to the target medical claim history based on similarity, frequency, and recency; generates a similarity score, a frequency score, and a recency score for each of the plurality of users; and selects the users to be included in the targeted subset based on the similarity score, the frequency score, and the recency score. The processor then monitors, for a period of time, the target medical claim history in comparison with medical claim histories to detect an anomaly in the target medical claim history; and causes an electronic communication to be displayed by a client device that comprises initiation of an intervention to be performed. Other embodiments are disclosed herein.

### BACKGROUND

Since a user's perception of an organization can be greatly influenced by the customer service that is provided to the user, the organization has interest in ensuring that the user's experience with the customer service is impeccable. Whether the user is interacting with a human agent or an automated agent, customer service aims to help the user complete his transaction in the most timely and efficient manner while ensuring that the user's experience with the customer service is enjoyable.

A user's experience is also further optimized when the user's needs or questions for customer service are anticipated such that the user does not have to reach out to customer service. For example, the Medicare-eligible population which includes users of 65 years of age or older can greatly benefit from having their needs or questions anticipated given their potential limitations in technological skills, physical mobility and access to assistance.

### BRIEF DESCRIPTION OF THE SEVERAL VIEWS OF THE DRAWINGS

In the drawings, which are not necessarily drawn to scale, like numerals may describe similar components in different views. To easily identify the discussion of any particular element or act, the most significant digit or digits in a reference number refer to the figure number in which that element is first introduced. Some embodiments are illustrated by way of example, and not limitation, in the figures of the accompanying drawings in which:

FIG. 1 is a block diagram showing an example system 100 including a targeted medical intervention system 110 according to various exemplary embodiments.

FIG. 2 is block diagram illustrating further details regarding the targeted medical intervention system 110, according to exemplary embodiments.

FIG. 3 is a schematic diagram illustrating data which may be stored in the database 208 of the targeted medical intervention system 110, according to various exemplary embodiments.

FIG. 4 illustrates a targeted medical intervention process 400 in accordance with one embodiment.

FIG. 5 illustrates details of the operation 404 of the process 400 in accordance with one embodiment.

FIG. 6 is a block diagram showing a software architecture within which examples may be implemented.

FIG. 7 is a diagrammatic representation of a machine in the form of a computer system within which a set of instructions may be executed for causing the machine to perform any one or more of the methodologies discussed herein, in accordance with some examples.

### DETAILED DESCRIPTION

The description that follows includes systems, methods, techniques, instruction sequences, and computing machine program products that embody illustrative embodiments of the disclosure. In the following description, for the purposes of explanation, numerous specific details are set forth in order to provide an understanding of various embodiments of the inventive subject matter. It will be evident, however, to those skilled in the art, that embodiments of the inventive subject matter may be practiced without these specific details. In general, well-known instruction instances, protocols, structures, and techniques are not necessarily shown in detail.

Among other things, embodiments of the present disclosure improve the functionality of customer service methods and systems by implementing a targeted medical intervention system that causes an initiation of an intervention for a target user based on a detection of an anomaly in the target user's medical claim history in comparison to the medical claims histories of a targeted subset of the population of users that is similar to the target user.

A user, e.g., a member or a representative of the member, may access the customer service of the organization. The user may contact customer service to accomplish a variety of tasks that can greatly vary in complexity. For example, simple tasks include getting assistance in registering for a username and password on the organization's website, or resetting a password associated with an online account while more complex tasks include checking an order status, placing a refill order, requesting information regarding a prescription, or requesting an explanation of benefits and terms associated with an account.

The organization can be a medical group that include members, people who benefit from the medical group or are provided with medical treatment by the group. The medical group can be a medical insurer or a pharmacy benefit manager (PBM), for example. The PBM may store data regarding member usage of prescription drugs. This data may be leveraged in order to provide a member the prescription coverage benefit and may be paid for by a client of the PBM. The clients of the PBM can include employers, group purchasing organizations, medical health plans and governmental groups. In general, prescription drug and medicine data may be accessed from a PBM database.

The organization can also provide its customers or members with access to customer service via different communication channels including audio calls (e.g., telephone call, VoIP, audio message, etc.) or via electronic messages (e.g., online chat, instant messaging, email, etc.).

While accessing customer service via telephone or electronic communications may be simple for most users, certain users may find themselves struggling to communicate with customer service due to age (e.g., Medicare-eligible population), lack of technological skills or access to technology, or due to difficulties in their life (e.g., loss of job, depression, loss of a family member, etc.). Accordingly, relying on the users to reach out to customer service when they are experiencing health or other challenges may result in the organization not being able to appropriately intervene with these users to rectify the situation.

Embodiments of the present disclosure aims to further optimize the customer service by anticipating which users need further interventions such as getting a follow-up telephone call or email to discuss the user's needs, when to reach out to these users, and further assessing whether additional interventions are needed.

Specifically, embodiments of the present disclosure implement a targeted medical intervention system by first generating a targeted subset of the users in a population that can be used to assess a target user. The population can include all the members in the customer base, all the members of a specific client, or all the members in a particular health plan, for example. Generating the targeted subset can be based on a number of indicators such as age, gender, medical claims history, marital status, socio-economic factors, and family history assessment. Once the targeted subset is obtained, anomalies in the medical claims history of the target user can be quickly detected and analyzed to determine whether an intervention is needed.

### Networked Computing Environment

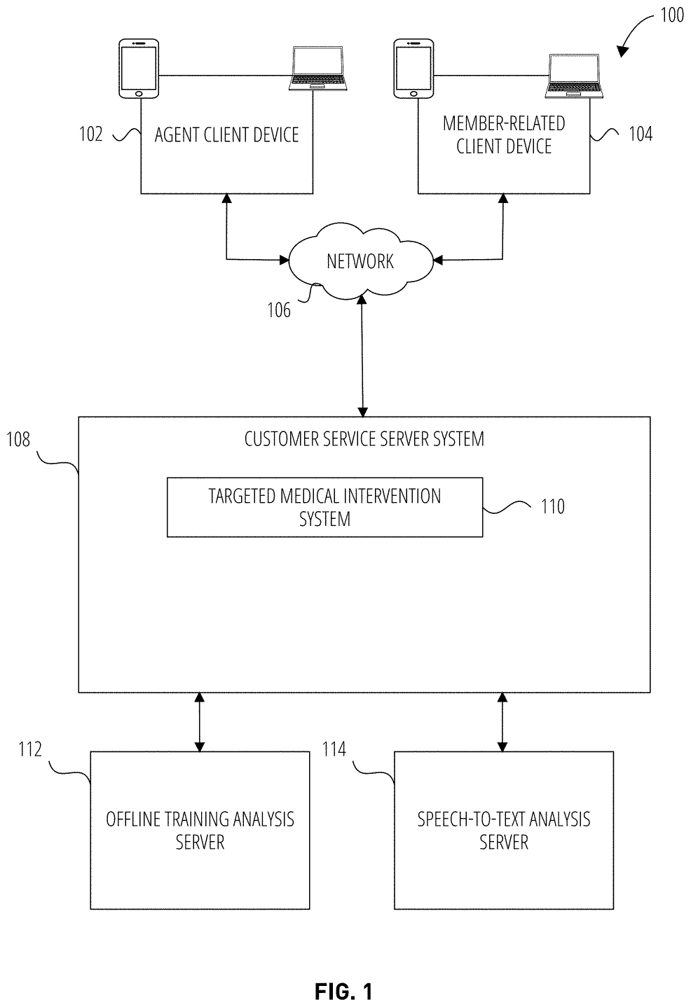

FIG. 1 is a block diagram showing an example system 100 according to various exemplary embodiments. The system 100 can be a customer service system that includes a customer service server system 108, an agent client device 102, and a member-related client device 104 that are communicatively coupled over a network 106 (e.g., Internet, telephony network, electronic communication network or the like).

The agent client device 102 and the member-related client device 104 can be communicatively coupled via an audio call (e.g., VoIP, Public Switched Telephone Network, cellular communication network, etc.) or via electronic messages (e.g., online chat, instant messaging, text messaging, email, and the like). In another embodiment, the agent client device 102 and the member-related client device 104 are communicatively coupled via a voice call, e.g., using a telephony network 106. While FIG. 1 illustrates a single agent client device 102 and a single member-related client device 104, it is understood that multiple agent client devices 102 and multiple member-related client devices 104 can be included in the system 100 in other embodiments. As used herein, the term “client device” may refer to any machine that interfaces to a communications network (such as network 106) to obtain resources from one or more server systems or other client devices. A client device may be, but is not limited to, a mobile phone, desktop computer, laptop, portable digital assistants (PDAs), smart phones, a wearable device (e.g., a smart watch), tablets, ultrabooks, netbooks, laptops, multi-processor systems, microprocessor-based or programmable consumer electronics, game consoles, set-top boxes, or any other communication device that a user may use to access a network. The member-related client device 104 can include a microphone and speaker on a mobile electronic device, a telephone, or a self-service kiosk, e.g., at a pharmacy, a clinic, a doctor's office, a mobile relief center, and the like. The member-related client device 104 can also include telecommunications devices for the deaf (TDD).

The network 106 may include, or operate in conjunction with, an ad hoc network, an intranet, an extranet, a virtual private network (VPN), a local area network (LAN), a wireless network, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, Gobi™, Bluetooth™, another type of network, or a combination of two or more such networks. For example, a network or a portion of a network may include a wireless or cellular network and the coupling may be a Code Division Multiple Access (CDMA) connection, a Global System for Mobile communications (GSM) connection, or other type of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

In the example shown in FIG. 1, a user using the member-related client device 104 can establish a communication session with an agent associated with the agent client device 102. The agent can be a human agent or an automated agent, e.g., on behalf of an organization. The automated agent can be associated with a medical group that includes the member. The automated agent can be an interactive voice response (IVR), a virtual online assistant, or a chatbot.

In another example, the agent using the agent client device 102 can establish a communication session with the user using the member-related client device 104. The agent using the agent client device 102 can also interact with the customer service server system 108 in order to train the neural networks in the customer service server system 108 which includes a targeted medical intervention system 110.

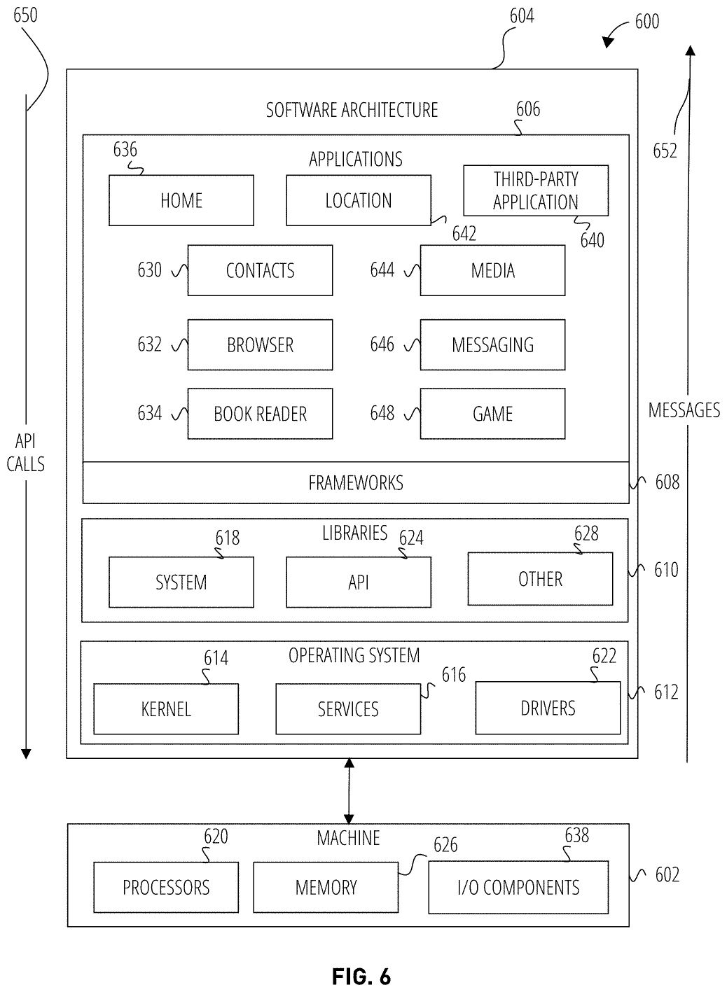

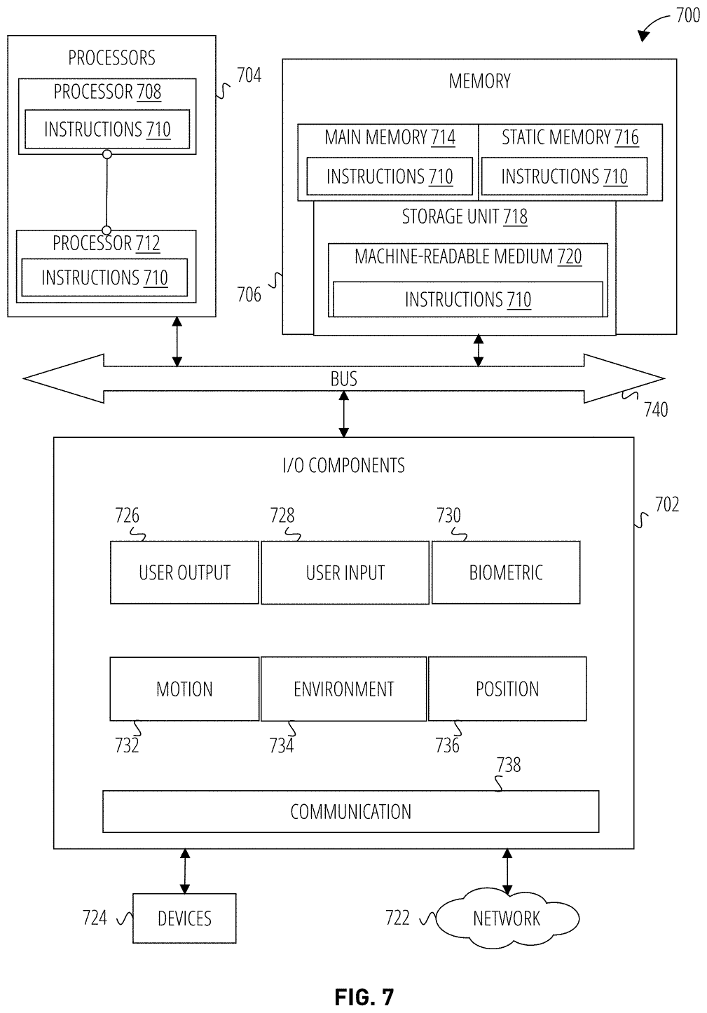

The customer service server system 108 can further include elements described with respect to FIG. 6 and FIG. 7, such as a processor and memory, having instructions stored thereon, that when executed by the processor, causes the processor to control the functions of the customer service server system 108.

As shown in FIG. 1, the system 100 can also include an offline training analysis server 112 and a speech-to-text analysis server 114 that are communicatively coupled to each other and to the customer service server system 108. In one embodiment, the offline training analysis server 112 and the speech-to-text analysis server 114 are included in the customer service server system 108 and communicatively coupled to the targeted medical intervention system 110.

In one embodiment, the offline training analysis server 112 can communicate with the targeted medical intervention system 110 to configure or execute the training tools for the neural networks in the targeted medical intervention system 110. Examples of training tools include the captcha human perception trainer and the medication image trainer further described herein.

### Targeted Medical Intervention System

In the customer service server system 108 of FIG. 1, the targeted medical intervention system 110 implements a data predictive model to timely intervene with a target user (e.g., a member) of the system 100 to help improve their overall health. One or more neural networks in the targeted medical intervention system 100 can be used to implement the data predictive model in order to make the determination as to whether an intervention is warranted. In one example, the target user can be a user who may be 65 years old or order, or Medicare-eligible. In another example, the target user can be any member of a medical group having access to the system 100.

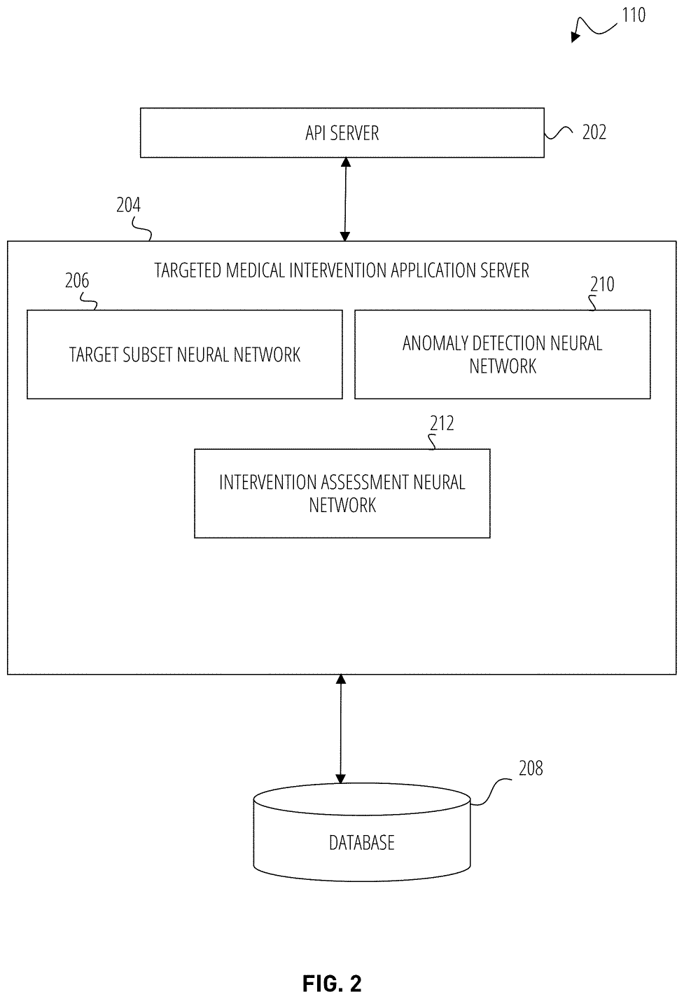

FIG. 2 is a block diagram illustrating further details regarding the targeted medical intervention system 110, according to exemplary embodiments. The targeted medical intervention system 110 includes an API server 202 that is coupled to and provides a programmatic interface to a targeted medical intervention application server 204. For example, the targeted medical intervention application server 204, using the API server 202, receive communications from the agent client device 102 or the member-related client device 104, commands or training session data to provision the neural networks in the targeted medical intervention system 110 from the agent client device 102, etc.

As shown in FIG. 2, the targeted medical intervention application server 204 includes a target subset neural network 206, an anomaly detection neural network 210, and an intervention assessment neural network 212. The target subset neural network 206 can receive indicators including age, gender, medical claims history, marital status, socio-economic factors, and family history assessment, and generate a targeted subset from the plurality of users for a target user. In one example, the target subset neural network 206 can generate a plurality of targeted subsets, each targeted subset being associated with a different target user. For example, a first targeted subset can include users aged 65 years and older, having diabetes, and recently widowed and a second targeted subset can include users aged between 50 and 64 years, on medication for depression, and having recently lost a family member.

The anomaly detection neural network 210 receives the targeted subsets that are generated by the target subset neural network 206 and receives the medical claims history of each user in the targeted subset. For each of the targeted groups, individuals within the targeted group can be a target user where anomalies in their medical claim history can be detected in relation to the medical claim histories of the other users in the targeted group. The anomaly detection neural network 210 detects and identifies the anomalies in the medical claims histories that may be cause for intervention.

The intervention assessment neural network 212 receives the targeted subsets from the target subset neural network 206 and the anomalies from the anomaly detection neural network 210 and determines whether an intervention is warranted. The intervention assessment neural network 212 can further use the indicators from the target subset neural network 206 to make the determination of whether the intervention is warranted. In an example embodiment, the neural networks can include a Gated Recurrent Unit (GRU) neural network.

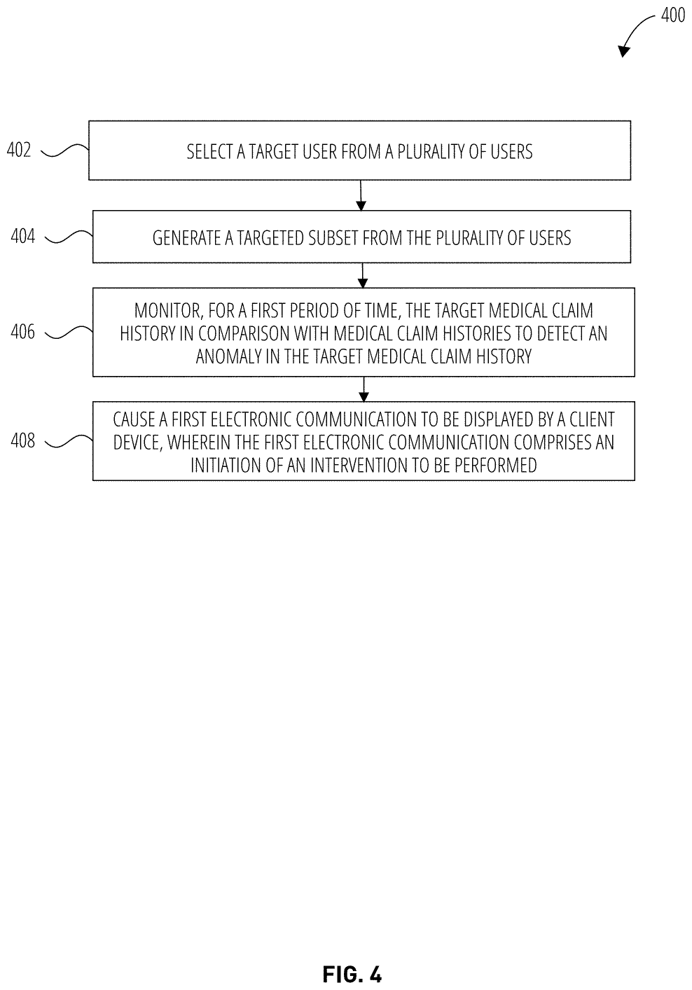

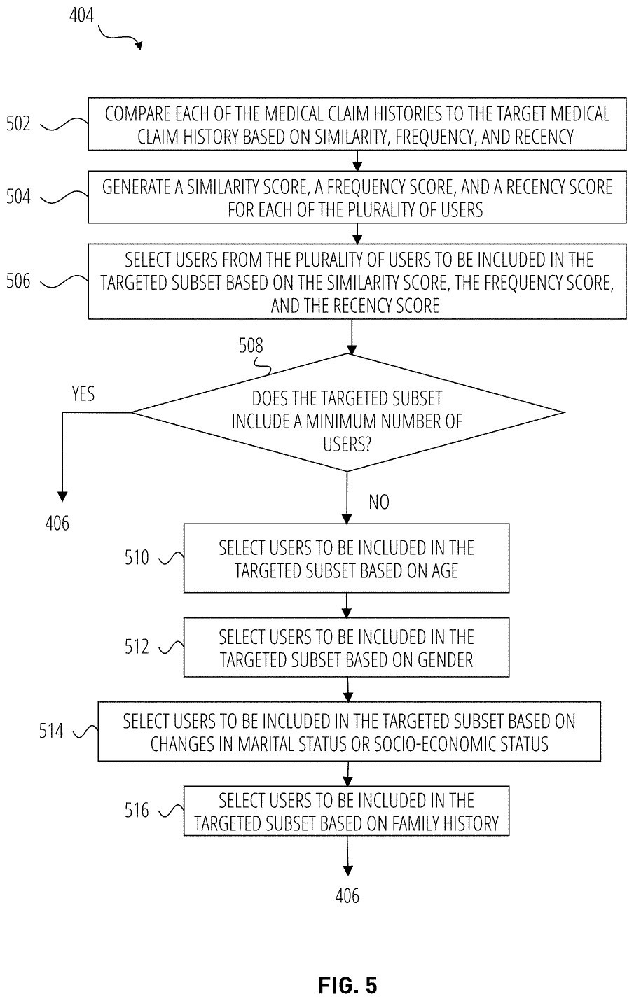

The targeted medical intervention application server 204 is communicatively coupled to the database 208, in which is stored data processed by the targeted medical intervention application server 204, as further described herein. In one embodiment, rather than including neural networks, the targeted medical intervention application server 204 includes a memory that stores instructions that, when executed by a processor, causes the processor to perform the operations of the targeted medical intervention application server 204. For example, the processor can perform the operations of process 400 in FIG. 4 and operation 404 in FIG. 5. While the example in FIG. 2 illustrates neural networks, other data analytics tools can be used in addition or in place of the neural networks.

### Database

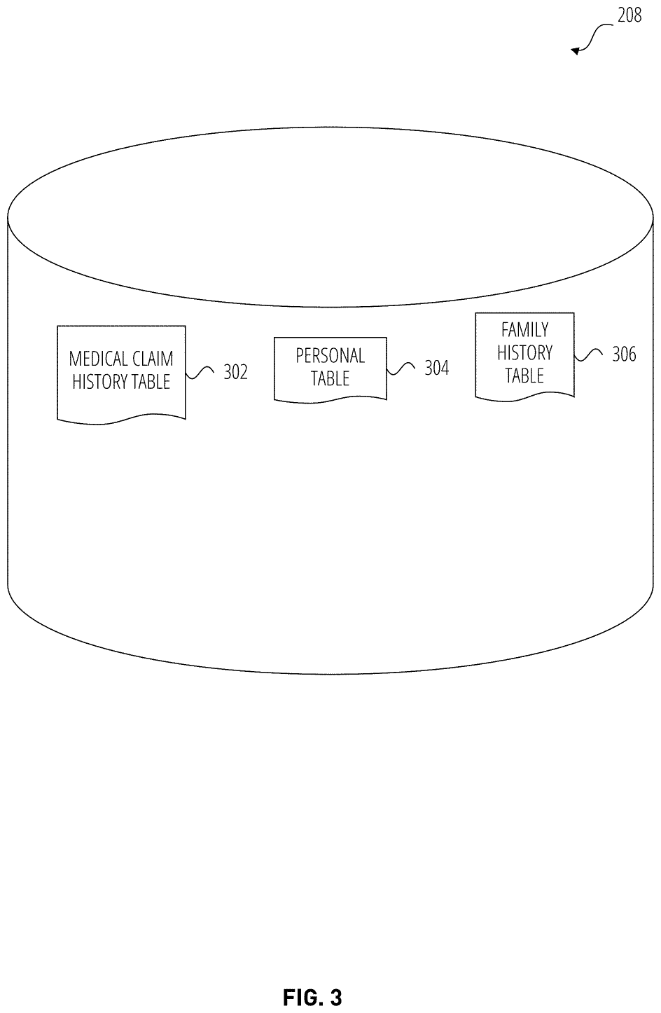

FIG. 3 is a schematic diagram illustrating data that is stored in the database 208 of the targeted medical intervention system 110, according to certain exemplary embodiments. While the content of the database 208 is shown to comprise a number of tables, the data could be stored in other types of data structures (e.g., as an object-oriented database). The database 208 includes a medical claim history table 302, a personal table 304, and a family history table 306. In one embodiment, the medical claim history table 302, personal table 304, and family history table 306 are updated in real-time such that these databases comprise real-time data. In another embodiment, the tables in the database 208 are updated periodically.

The medical claim history table 302 can store the medication claims histories of each of the users in a population related to the system 100. The population can include all the members in the customer base of the system 100 (e.g., all the employees of all the companies that are customers of an organization or medical group), all the members of a specific client (e.g., all the employees of one company that is a customer of the organization or medical group), or all the members in a particular health plan (e.g., all employees subscribed to a given health plan offered by the medical group), for example. For example, the medical claim history table 302 can store the history of medical claims and prescription claims including dates of each claim, the costs of each claim, the payment history, the medication identifier, the medical procedures, identification of the medical practitioner (e.g., the prescribing doctor or pharmacist), the order number, the prescription number, etc.

The personal table 304 can store, for each user, personal information including the user's name, address, phone number, email address, employer, employment status, socio-economic status, medications prescribed to the user, medical conditions attributed to the user, marital status, medical group plan identifier, identification of the user's medical professionals, etc.

The family history table 306 stores, for each user, the family history assessment that is available. For example, the family history table 306 can store the medical history of the user's family members, treatments the user's family members are undergoing or previously have undergone, changes in socio-economic status for the user's family members, etc.

While not shown in FIG. 3, an association table can be included in database 208 to maintain the associations between the tables in the database 208 or between the elements within a single table in the database 208. For example, the association table can include pointers from each patient (e.g., each of the users in a population related to the system 100) to the patients in the determined comparative group (e.g., target subset of users). In one example, the association table can be updated periodically such that the pointers would be updated based on the updated target subset of users for each of the users in the population related to the system 100. In one example, the pointers in the association table are associated with a time stamp that identifies the time or date at which the pointers were last updated. For example, using the time stamps, the targeted medical intervention system 110 can determine when the targeted subset of users associated with a given user was reassessed. For each of the users being added or removed from the updated targeted subset of users, the targeted medical intervention system 110 can store information (e.g., delta) related to the decision to add or remove the user from the updated targeted subset of users. In one example, the targeted medical intervention system 110 can further implement weights based on the number of time stamps associated with a pointer such that the greater the number of time stamps can be allocated greater weighting during relative comparisons.

Although the described flowcharts can show operations as a sequential process, many of the operations can be performed in parallel or concurrently. In addition, the order of the operations may be re-arranged. A process is terminated when its operations are completed. A process may correspond to a method, a procedure, an algorithm, etc. The operations of methods may be performed in whole or in part, may be performed in conjunction with some or all of the operations in other methods, and may be performed by any number of different systems, such as the systems described herein, or any portion thereof, such as a processor included in any of the systems, such as the systems described in FIG. 1 and/or FIG. 7.

### Targeted Medical Intervention Process

FIG. 4 is a flowchart of an exemplary targeted medical intervention process 400 according to various aspects of the disclosure. The process 400 can be performed by the targeted medical intervention system 110 of the customer service server system 108 in FIG. 1. In one embodiment, a processor (or circuitry dedicated to performing instructed tasks) included in the customer service server system 108 performs the process 400 or causes the customer service server system 108 to perform the process 400.

Process 400 starts, at operation 402, with a processor selecting a target user from a plurality of users that are associated with a plurality of medical claim histories. For instance, the target user is associated with a target medical claim history that is included in the plurality of medical claim histories stored in the database 208. The medical claims histories can also include the prescription drug claims. In one example, the target user can be part of a user population of a particular client and/or health plan of which the target user is a member.

At operation 404, the processor generates a targeted subset from the plurality of users. The targeted subset is a group of individuals that are similar to the target user based on indicators such as age, gender, medical claims history, marital status, socio-economic factors, and family history assessment. In one example, the targeted subset can be used by a person (e.g., health care provider or administrator) who wishes to monitor the health of a particular person (e.g., the target user), or a set of persons (e.g., targeted subset) with a particular health condition (e.g., high blood pressure, cancer, persons taking more than 5 prescription drugs, persons over a particular age, etc.).

In one example, the processor uses the user association table included in the database that associates the target user with the targeted subset. The association table may include links or pointers that associates a targeted user object with the user objects of the remaining subset. In some examples, each link may be associated with a timestamp that indicates the time or date at which each of the users in the targeted subset were added or removed from the targeted subset. In addition, the targeted subset may be at least substantially unique for each target user. For example, each user of the targeted subset does not have to be in a respective targeted subset. Accordingly, data privacy and data integrity can be better maintained and data change patterns may be easier to detect.

Once the targeted subset is generated, at operation 406, the processor monitors, for a first period of time, the target medical claim history of the target user in comparison with medical claim histories of the users in the targeted subset to detect an anomaly in the target medical claim history. For example, the first period of time can be a monitoring period that is established to be a month. In this example, the processor monitors the target medical claim history and the medical claim histories associated with the users in the targeted subset by measuring the difference in the number of medical claims associated with the targeted group compared to the target user. The processor can also group the claims in the medical claim histories associated with the users in the targeted subset, map the claims to the relevant medical conditions, and further assess the target user's condition with that of the targeted subset. Using this mapping, the processor can determine whether the target user has a same condition that was previously known from his medical claim history or a new condition. The processor can also determine whether the target user has a more serious condition or is showing an improvement in his condition compared to the targeted subset. In another example, in addition or in lieu of the medical claims, the processor can monitor a history of prescription claims and other types of claims of the target user in comparison with a history of the prescription claims and other types of claims of the target subset, at operation 406, in order to detect an anomaly in the prescription claims and other types of claims.

In some embodiments, monitoring of the claim history of the user relative to the claim histories of the members of the targeted subset may include calculating one or more than one of the following factors: (i) type of claim (e.g., medical claim, pharmacy claim, dental claim, etc.), (ii) number of claims received in a time period, (iii) whether claims are associated with an existing health condition or a new health condition, (iv) the nature of care received associated with a claim (e.g., emergency room care, virtual care, office visit, home visit, etc.), (v) frequency of care, and (vi) provider variance (e.g., number of different providers seen for the same type of care in the same or different care episodes associated with the health condition). The processor can thus gather insights based on these factors in operation 406. In one example, the processor's data monitoring, measurement, and comparison searches for statistical differences between the data of target user and the users in the target subset to identify a change in the claims history.

The processor can then use the insights from the monitoring in operation 406 and from an analysis of the data in the database 208 to determine whether to cause an initiation of an intervention with the target user due to the anomaly that is detected. For instance, the processor can determine whether to cause the initiation of the intervention based on changes in the target medical claim history, changes in marital status of the target user, change in socio-economic status of the target user, family history of the target user, or any combination thereof. In one example, the data pertaining to the marital status, socio-economic status, family history, etc, can be collected by the system 100, purchased from third-party companies external to the system 100, provided by the users of system 100, gathered from social media networks, or otherwise. Accordingly, the interventions can be made based on a delta that the target user exhibits in comparison to the targeted subset.

Alternatively, the interventions can be made for the entirety of the group that is shown to be experiencing the same set of circumstances. For example, if the target user is a diabetic patient receiving interventions to manage diabetes and the target user no longer has access to healthy food because a neighborhood grocery store closes, the target user in addition to the users in the targeted group (e.g., diabetic users of an age range, living alone within a geographical location) may all benefit from an intervention that leads to organized group transport to the grocery store or grocery home delivery.

When an intervention is determined to be warranted, at operation 408, the processor causes a first electronic communication to be displayed by a client device that comprises an initiation of an intervention to be performed. The first electronic communication can be a text message (e.g., SMS), an email, or a chat message or notification that is caused to be displayed by the agent client device 102 or to the member-related client device 104. In another embodiment, the first electronic communication can include an active link or URL, or selectable icon or text that can be activated by the recipient (e.g., the agent or the user) to establish a communication session between the agent and the user. The intervention can comprise an audio call (e.g., VoIP, Public Switched Telephone Network, cellular communication network, etc.) or via electronic messages (e.g., online chat, instant messaging, text messaging, email, and the like). The invention can also be designed to address medical, behavioral, or other needs such as coordinating rides to the doctor appointments, providing housing assistance, delivering groceries, providing information on support groups, and other resources available in a particular community. The electronic communication can, for example, automatically cause the initiation of the intervention (e.g., switching the target user to prescription home delivery, sending the sign-up information for support groups, coordinating the transportation to upcoming doctor appointments, etc.).

In one example, the electronic communication can establish a communication session with a human agent or an automated agent that assesses the target user's medical, behavioral, or other needs and determines, based on the responses and the indicators associated with the target user, the type of intervention that is best suited to the target user. The processor can also determine based on the indicators associated with the target user the most effective or appropriate manner of making the intervention (e.g., dispatching an agent for home visit, triggering an automated phone call, scheduling a video conference with a human agent, sending text message, email, etc.).

Additionally, the processor can implement follow up assessments over a second period of time to determine if the intervention was successful or needs to be adjusted. Accordingly, the processor can further monitor, for a second period of time, the target medical claim history in comparison with medical claim histories to detect changes in the target medical claim history. For example, the processor can monitor for a subsequent month, the target medical claim history in comparison with medical claim histories of the targeted subset to determine if any further anomalies are detected or if the anomaly previously detected has decreased (e.g., number of claims reduced from the higher anomalous level). To further inform the follow up assessment, the processor can receive the information from the target user (e.g., self-assessment) or can further review the indicators over the second period of time.

In one example, the processor can determine a success score of the intervention based on the changes in the target medical claims history detected during the second period of time. The processor can further cause a second electronic communication to be displayed by the client device that comprises an initiation of a subsequent intervention to be performed. For example, the processor can generate a second electronic communication to be displayed by the client device that includes the adjustments to be made to the intervention that was determined not to be effective. Adjustments can also be made to account for the changes in the target user's medical, behavioral or other changes in their environment. In one example, the second electronic communication can be a signal that causes the adjustments to be automatically made (e.g., switching the target user to prescription home delivery and sending this change notification to the target user).

### Process of Generating a Targeted Subset

FIG. 5 illustrates details of generating the targeted subset in operation 404 of the process 400 in accordance with one embodiment. The operation 404 in FIG. 5 can be performed by the targeted medical intervention system 110 of the customer service server system 108 in FIG. 1. In one embodiment, a processor (or circuitry dedicated to performing instructed tasks) included in the customer service server system 108 performs the operations in FIG. 5 or causes the customer service server system 108 to perform the operations in FIG. 5.

As shown in FIG. 5, to generate the targeted subset, the processor can start at operation 502 by comparing each of the medical claim histories of the plurality of users in the population to the target medical claim history of the target user based on similarity, frequency, and recency. For example, if the target user has diabetes, has weekly claims for the treatments and prescriptions, the processor assesses the similarity of the medical claims by finding claims for diabetes treatments and prescriptions claims for diabetes medication, analyzes the frequency of the medical claims to find other users that have similar weekly claims, and finally assesses the recency of the claims by finding users having recent claims for diabetes treatments and prescriptions claims for diabetes medication. At operation 504, the processor generates a similarity score, a frequency score, and a recency score for each of the plurality of users, and at operation 506, the processor selects users from the plurality of users to be included in the targeted subset based on the similarity score, the frequency score, and the recency score. Each of the scores can be weighted such that the processor may select the users to be included in the targeted subset based on a plurality of weights associated with the similarity score, the frequency score, and the recency score.

At operation 508, the processor determines if the targeted subset includes a minimum number of users. In one example, the targeted subset is a small group of 20-25 users. In another examples, the minimum number of users in a targeted subset can vary based on the indicators associated with the targeted subset. The processor can determine if the targeted subset includes a minimum number of users by comparing the number of users obtained in the targeted subset after operation 506 to a predetermined threshold that is established to be the desired minimum number of users for the targeted subset.

When the processor determines at operation 508 that the targeted subset includes a minimum number of users, the process proceeds to operation 406 in FIG. 4. When the processor determines, at operation 508, that the targeted subset does not include a minimum number of users, the processor, at operation 510, selects the users to be included in the targeted subset based on ages of the plurality of users and an age of the target user. For example, the processor can select additional users for the targeted subset that are within a predetermined age range from the target user. In another example, the processor generates an age score based on the difference in age of each of the users to the target user. For instance, the score increases exponentially as the difference in age increases. A plurality of scores can also be associated to different buckets of age ranges. In this example, the higher the score allocated to the user's age the least likely the user is determined to be similar to the target user and will not be added to the targeted subset.

At operation 512, the processor can select the users to be included in the targeted subset based on genders of the plurality of users and a gender of the target user. Similarly, to age, the processor can generate gender scores where the user is associated with a low score (e.g., 0) if the user is the same gender as the target user, and with a high score (e.g., 10) if the user is a different gender from the target user. In this example, the higher the score allocated to the user's gender the least likely the user is determined to be similar to the target user and will not be added to the targeted subset.

In one example, the processor can detect changes in marital status or socio-economic status associated with the plurality of users and a change in marital status or socio-economic status associated with the target user. In this example, at operation 514, the processor can select the users to be included in the targeted subset based on the changes in marital status or socio-economic status associated with the plurality of users and the change in marital status or socio-economic status associated with the target user. The processor can also generate scores for marital status and socio-economic status that are low when the changes in marital status and socio-economic status are the same or similar to the target user and high when the changes are different from the target user. In this example, the higher the score allocated to the user's changes in marital status and socio-economic status the least likely the user is determined to be similar to the target user and will not be added to the targeted subset.

At operation 516, the processor can perform an aggregate family history assessment for each of the plurality of users and select the users to be included in the targeted subset based on the aggregate family history assessment. For example, if the user and the target user are associated with the recent loss of a family member, the processor can generate a score (e.g., low) that indicates a similarity between the user and the target user such that the user can be added to the targeted subset.

In one example, the processor selects the users to be included in the targeted subset based on an analysis of the scores generated in operations 510, 512, 514, and 516. For instance, the users associated with the lowest aggregated scores are added to the targeted subset. Having generated the targeted subset, the processor then proceeds to the operation 406.

### Software Architecture

FIG. 6 is a block diagram 600 illustrating a software architecture 604, which can be installed on any one or more of the devices described herein. The software architecture 604 is supported by hardware such as a machine 602 that includes processors 620, memory 626, and I/O components 638. In this example, the software architecture 604 can be conceptualized as a stack of layers, where each layer provides a particular functionality. The software architecture 604 includes layers such as an operating system 612, libraries 610, frameworks 608, and applications 606. Operationally, the applications 606 invoke API calls 650 through the software stack and receive messages 652 in response to the API calls 650.

The operating system 612 manages hardware resources and provides common services. The operating system 612 includes, for example, a kernel 614, services 616, and drivers 622. The kernel 614 acts as an abstraction layer between the hardware and the other software layers. For example, the kernel 614 provides memory management, processor management (e.g., scheduling), component management, networking, and security settings, among other functionalities. The services 616 can provide other common services for the other software layers. The drivers 622 are responsible for controlling or interfacing with the underlying hardware. For instance, the drivers 622 can include display drivers, camera drivers, BLUETOOTH® or BLUETOOTH® Low Energy drivers, flash memory drivers, serial communication drivers (e.g., USB drivers), WI-FI® drivers, audio drivers, power management drivers, and so forth.

The libraries 610 provide a common low-level infrastructure used by the applications 606. The libraries 610 can include system libraries 618 (e.g., C standard library) that provide functions such as memory allocation functions, string manipulation functions, mathematic functions, and the like. In addition, the libraries 610 can include API libraries 624 such as media libraries (e.g., libraries to support presentation and manipulation of various media formats such as Moving Picture Experts Group-4 (MPEG4), Advanced Video Coding (H.264 or AVC), Moving Picture Experts Group Layer-3 (MP3), Advanced Audio Coding (AAC), Adaptive Multi-Rate (AMR) audio codec, Joint Photographic Experts Group (JPEG or JPG), or Portable Network Graphics (PNG)), graphics libraries (e.g., an OpenGL framework used to render in two dimensions (2D) and three dimensions (3D) in a graphic content on a display), database libraries (e.g., SQLite to provide various relational database functions), web libraries (e.g., WebKit to provide web browsing functionality), and the like. The libraries 610 can also include a wide variety of other libraries 628 to provide many other APIs to the applications 606.

The frameworks 608 provide a common high-level infrastructure that is used by the applications 606. For example, the frameworks 608 provide various graphical user interface (GUI) functions, high-level resource management, and high-level location services. The frameworks 608 can provide a broad spectrum of other APIs that can be used by the applications 606, some of which may be specific to a particular operating system or platform.

In an example, the applications 606 may include a home application 636, a contacts application 630, a browser application 632, a book reader application 634, a location application 642, a media application 644, a messaging application 646, a game application 648, and a broad assortment of other applications such as a third-party application 640. The applications 606 are programs that execute functions defined in the programs. Various programming languages can be employed to create one or more of the applications 606, structured in a variety of manners, such as object-oriented programming languages (e.g., Objective-C, Java, or C++) or procedural programming languages (e.g., C or assembly language). In a specific example, the third-party application 640 (e.g., an application developed using the ANDROID™ or IOS™ software development kit (SDK) by an entity other than the vendor of the particular platform) may be mobile software running on a mobile operating system such as IOS™, ANDROID™, WINDOWS® Phone, or another mobile operating system. In this example, the third-party application 640 can invoke the API calls 650 provided by the operating system 612 to facilitate functionality described herein.

### Machine Architecture

FIG. 7 is a diagrammatic representation of the machine 700 within which instructions 710 (e.g., software, a program, an application, an applet, an app, or other executable code) for causing the machine 700 to perform any one or more of the methodologies discussed herein may be executed. For example, the instructions 710 may cause the machine 700 to execute any one or more of the methods described herein. The instructions 710 transform the general, non-programmed machine 700 into a particular machine 700 programmed to carry out the described and illustrated functions in the manner described. The machine 700 may operate as a standalone device or may be coupled (e.g., networked) to other machines. In a networked deployment, the machine 700 may operate in the capacity of a server machine or a client machine in a server-client network environment, or as a peer machine in a peer-to-peer (or distributed) network environment. The machine 700 may comprise, but not be limited to, a server computer, a client computer, a personal computer (PC), a tablet computer, a laptop computer, a netbook, a set-top box (STB), a personal digital assistant (PDA), an entertainment media system, a cellular telephone, a smartphone, a mobile device, a wearable device (e.g., a smartwatch), a smart home device (e.g., a smart appliance), other smart devices, a web appliance, a network router, a network switch, a network bridge, or any machine capable of executing the instructions 710, sequentially or otherwise, that specify actions to be taken by the machine 700. Further, while only a single machine 700 is illustrated, the term “machine” shall also be taken to include a collection of machines that individually or jointly execute the instructions 710 to perform any one or more of the methodologies discussed herein. The machine 700, for example, may comprise the agent client device 102 or any one of a number of server devices in customer service server system 108. In some examples, the machine 700 may also comprise both client and server systems, with certain operations of a particular method or algorithm being performed on the server-side and with certain operations of the particular method or algorithm being performed on the client-side.

The machine 700 may include processors 704, memory 706, and input/output I/O components 638, which may be configured to communicate with each other via a bus 740. In an example, the processors 704 (e.g., a Central Processing Unit (CPU), a Reduced Instruction Set Computing (RISC) Processor, a Complex Instruction Set Computing (CISC) Processor, a Graphics Processing Unit (GPU), a Digital Signal Processor (DSP), an Application Specific Integrated Circuit (ASIC), a Radio-Frequency Integrated Circuit (RFIC), another processor, or any suitable combination thereof) may include, for example, a processor 708 and a processor 712 that execute the instructions 710. The term “processor” is intended to include multi-core processors that may comprise two or more independent processors (sometimes referred to as “cores”) that may execute instructions contemporaneously. Although FIG. 7 shows multiple processors 704, the machine 700 may include a single processor with a single-core, a single processor with multiple cores (e.g., a multi-core processor), multiple processors with a single core, multiple processors with multiples cores, or any combination thereof.

The memory 706 includes a main memory 714, a static memory 716, and a storage unit 718, both accessible to the processors 704 via the bus 740. The main memory 706, the static memory 716, and storage unit 718 store the instructions 710 embodying any one or more of the methodologies or functions described herein. The instructions 710 may also reside, completely or partially, within the main memory 714, within the static memory 716, within machine-readable medium 720 within the storage unit 718, within at least one of the processors 704 (e.g., within the Processor's cache memory), or any suitable combination thereof, during execution thereof by the machine 700.

The I/O components 702 may include a wide variety of components to receive input, provide output, produce output, transmit information, exchange information, capture measurements, and so on. The specific I/O components 702 that are included in a particular machine will depend on the type of machine. For example, portable machines such as mobile phones may include a touch input device or other such input mechanisms, while a headless server machine will likely not include such a touch input device. It will be appreciated that the I/O components 702 may include many other components that are not shown in FIG. 7. In various examples, the I/O components 702 may include user output components 726 and user input components 728. The user output components 726 may include visual components (e.g., a display such as a plasma display panel (PDP), a light-emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The user input components 728 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and force of touches or touch gestures, or other tactile input components), audio input components (e.g., a microphone), and the like.

In further examples, the I/O components 702 may include biometric components 730, motion components 732, environmental components 734, or position components 736, among a wide array of other components. For example, the biometric components 730 include components to detect expressions (e.g., hand expressions, facial expressions, vocal expressions, body gestures, or eye-tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 732 include acceleration sensor components (e.g., accelerometer), gravitation sensor components, rotation sensor components (e.g., gyroscope).

The environmental components 734 include, for example, one or cameras (with still image/photograph and video capabilities), illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment.

The position components 736 include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 702 further include communication components 738 operable to couple the machine 700 to a network 722 or devices 724 via respective coupling or connections. For example, the communication components 738 may include a network interface component or another suitable device to interface with the network 722. In further examples, the communication components 738 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 724 may be another machine or any of a wide variety of peripheral devices (e.g., a peripheral device coupled via a USB).

Moreover, the communication components 738 may detect identifiers or include components operable to detect identifiers. For example, the communication components 738 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 738, such as location via Internet Protocol (IP) geolocation, location via Wi-Fi® signal triangulation, location via detecting an NFC beacon signal that may indicate a particular location, and so forth.

The various memories (e.g., main memory 714, static memory 716, and memory of the processors 704) and storage unit 718 may store one or more sets of instructions and data structures (e.g., software) embodying or used by any one or more of the methodologies or functions described herein. These instructions (e.g., the instructions 710), when executed by processors 704, cause various operations to implement the disclosed examples.

The instructions 710 may be transmitted or received over the network 722, using a transmission medium, via a network interface device (e.g., a network interface component included in the communication components 738) and using any one of several well-known transfer protocols (e.g., hypertext transfer protocol (HTTP)). Similarly, the instructions 710 may be transmitted or received using a transmission medium via a coupling (e.g., a peer-to-peer coupling) to the devices 724.

### Glossary

“Carrier signal” refers to any intangible medium that is capable of storing, encoding, or carrying instructions for execution by the machine, and includes digital or analog communications signals or other intangible media to facilitate communication of such instructions. Instructions may be transmitted or received over a network using a transmission medium via a network interface device.

“Client device” refers to any machine that interfaces to a communications network to obtain resources from one or more server systems or other client devices. A client device may be, but is not limited to, a mobile phone, desktop computer, laptop, portable digital assistants (PDAs), smartphones, tablets, ultrabooks, netbooks, laptops, multi-processor systems, microprocessor-based or programmable consumer electronics, game consoles, set-top boxes, or any other communication device that a user may use to access a network.

“Communication network” refers to one or more portions of a network that may be an ad hoc network, an intranet, an extranet, a virtual private network (VPN), a local area network (LAN), a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network may include a wireless or cellular network and the coupling may be a Code Division Multiple Access (CDMA) connection, a Global System for Mobile communications (GSM) connection, or other types of cellular or wireless coupling. In this example, the coupling may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long-range protocols, or other data transfer technology.

“Component” refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein. A hardware component may also be implemented mechanically, electronically, or any suitable combination thereof. For example, a hardware component may include dedicated circuitry or logic that is permanently configured to perform certain operations. A hardware component may be a special-purpose processor, such as a field-programmable gate array (FPGA) or an application specific integrated circuit (ASIC). A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software), may be driven by cost and time considerations. Accordingly, the phrase “hardware component” (or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time. Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output. Hardware components may also initiate communications with input or output devices, and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (SaaS). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

“Computer-readable storage medium” refers to both machine-storage media and transmission media. Thus, the terms include both storage devices/media and carrier waves/modulated data signals. The terms “machine-readable medium,” “computer-readable medium” and “device-readable medium” mean the same thing and may be used interchangeably in this disclosure.

“Machine storage medium” refers to a single or multiple storage devices and media (e.g., a centralized or distributed database, and associated caches and servers) that store executable instructions, routines and data. The term shall accordingly be taken to include, but not be limited to, solid-state memories, and optical and magnetic media, including memory internal or external to processors. Specific examples of machine-storage media, computer-storage media and device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks The terms “machine-storage medium,” “device-storage medium,” “computer-storage medium” mean the same thing and may be used interchangeably in this disclosure. The terms “machine-storage media,” “computer-storage media,” and “device-storage media” specifically exclude carrier waves, modulated data signals, and other such media, at least some of which are covered under the term “signal medium.”

“Non-transitory computer-readable storage medium” refers to a tangible medium that is capable of storing, encoding, or carrying the instructions for execution by a machine.

“Signal medium” refers to any intangible medium that is capable of storing, encoding, or carrying the instructions for execution by a machine and includes digital or analog communications signals or other intangible media to facilitate communication of software or data. The term “signal medium” shall be taken to include any form of a modulated data signal, carrier wave, and so forth. The term “modulated data signal” means a signal that has one or more of its characteristics set or changed in such a matter as to encode information in the signal. The terms “transmission medium” and “signal medium” mean the same thing and may be used interchangeably in this disclosure.

What is claimed is:

1. A method comprising:
selecting, by a processor, a target user from a plurality of users, wherein the plurality of users are associated with a plurality of medical claim histories, wherein the target user is associated with a target medical claim history included in the plurality of medical claim histories;
generating, using a target subset neural network, a targeted subset from the plurality of users, wherein generating the targeted subset comprises:
comparing each of the medical claim histories to the target medical claim history based on similarity, frequency, and recency,
generating a similarity score, a frequency score, and a recency score for each of the plurality of users, and
selecting users from the plurality of users to be included in the targeted subset based on the similarity score, the frequency score, and the recency score;
monitoring, for a first period of time, using an anomaly detection neural network, the target medical claim history in comparison with medical claim histories associated with the users in the targeted subset to detect an anomaly in the target medical claim history; and
causing a first electronic communication to be displayed by a client device, wherein the first electronic communication comprises an initiation of an intervention to be performed.

2. The method of claim 1, wherein generating the targeted subset further comprises:
selecting the users to be included in the targeted subset based on a plurality of weights associated with the similarity score, the frequency score, and the recency score.

3. The method of claim 1, wherein generating the targeted subset further comprises:
selecting the users to be included in the targeted subset based on ages of the plurality of users and an age of the target user.

4. The method of claim 1, wherein generating the targeted subset further comprises:
selecting the users to be included in the targeted subset based on genders of the plurality of users and a gender of the target user.

5. The method of claim 1, wherein generating the targeted subset further comprises:
detecting changes in marital status or socio-economic status associated with the plurality of users and a change in marital status or socio-economic status associated with the target user, and
selecting the users to be included in the targeted subset based on the changes in marital status or socio-economic status associated with the plurality of users and the change in marital status or socio-economic status associated with the target user.

6. The method of claim 1, wherein generating the targeted subset further comprises:
performing an aggregate family history assessment for each of the plurality of users, and
selecting the users to be included in the targeted subset based on the aggregate family history assessment.

7. The method of claim 1, further comprising:
determining, using an intervention assessment neural network, whether to cause the initiation of the intervention based on changes in the target medical claim history, changes in marital status of the target user, change in socio-economic status of the target user, family history of the target user, or any combination thereof.

8. The method of claim 1, further comprising:
monitoring, for a second period of time, the target medical claim history in comparison with the medical claim histories of the users in the targeted subset to detect changes in the target medical claim history.

9. The method of claim 8, further comprising:
determining a success score of the intervention based on the changes in the target medical claims history.

10. The method of claim 9, further comprising:
causing a second electronic communication to be displayed by the client device, wherein the second electronic communication comprises an initiation of a subsequent intervention to be performed.

11. A non-transitory computer-readable storage medium, the computer-readable storage medium including instructions that when executed by a processor, cause the processor to perform operations comprising:
selecting a target user from a plurality of users, wherein the plurality of users are associated with a plurality of medical claim histories, wherein the target user is associated with a target medical claim history included in the plurality of medical claim histories;
generating, using a target subset neural network, a targeted subset from the plurality of users, wherein generating the targeted subset comprises:
comparing each of the medical claim histories to the target medical claim history based on similarity, frequency, and recency,
generating a similarity score, a frequency score, and a recency score for each of the plurality of users, and
selecting users from the plurality of users to be included in the targeted subset based on the similarity score, the frequency score, and the recency score;
monitoring, for a first period of time, using an anomaly detection neural network, the target medical claim history in comparison with medical claim histories associated with the users in the targeted subset to detect an anomaly in the target medical claim history; and
causing a first electronic communication to be displayed by a client device, wherein the first electronic communication comprises an initiation of an intervention to be performed.

12. The computer-readable storage medium of claim 11, wherein generating the targeted subset further comprises:
selecting the users to be included in the targeted subset based on a plurality of weights associated with the similarity score, the frequency score, and the recency score.

13. The computer-readable storage medium of claim 11, wherein generating the targeted subset further comprises:
selecting the users to be included in the targeted subset based on ages of the plurality of users and an age of the target user.

14. The computer-readable storage medium of claim 11, wherein generating the targeted subset further comprises:
selecting the users to be included in the targeted subset based on genders of the plurality of users and a gender of the target user.

15. The computer-readable storage medium of claim 11, wherein generating the targeted subset further comprises:
detecting changes in marital status or socio-economic status associated with the plurality of users and a change in marital status or socio-economic status associated with the target user, and
selecting the users to be included in the targeted subset based on the changes in marital status or socio-economic status associated with the plurality of users and the change in marital status or socio-economic status associated with the target user.

16. The computer-readable storage medium of claim 11, wherein generating the targeted subset further comprises:
performing an aggregate family history assessment for each of the plurality of users, and
selecting the users to be included in the targeted subset based on the aggregate family history assessment.

17. The computer-readable storage medium of claim 11, wherein the processor to perform operations further comprising:
Determining, using an intervention assessment neural network, whether to cause the initiation of the intervention based on changes in the target medical claim history, changes in marital status of the target user, change in socio-economic status of the target user, family history of the target user, or any combination thereof.

18. The computer-readable storage medium of claim 11, wherein the processor to perform operations further comprising:
monitoring, for a second period of time, the target medical claim history in comparison with the medical claim histories of the users in the targeted subset to detect changes in the target medical claim history.

19. The computer-readable storage medium of claim 18, wherein the processor to perform operations further comprising:
determining a success score of the intervention based on the changes in the target medical claims history.

20. The computer-readable storage medium of claim 19, wherein the processor to perform operations further comprising:
causing a second electronic communication to be displayed by the client device, wherein the second electronic communication comprises an initiation of a subsequent intervention to be performed.

21. A computing apparatus comprising:
a processor; and
a memory storing instructions that, when executed by the processor, causes the processor to perform operations comprising:
selecting a target user from a plurality of users, wherein the plurality of users are associated with a plurality of medical claim histories, wherein the target user is associated with a target medical claim history included in the plurality of medical claim histories;
generating, using a target subset neural network, a targeted subset from the plurality of users, wherein generating the targeted subset comprises:
comparing each of the medical claim histories to the target medical claim history based on similarity, frequency, and recency,
generating a similarity score, a frequency score, and a recency score for each of the plurality of users, and
selecting users from the plurality of users to be included in the targeted subset based on the similarity score, the frequency score, and the recency score;
monitoring, for a first period of time, using an anomaly detection neural network, the target medical claim history in comparison with medical claim histories associated with the users in the targeted subset to detect an anomaly in the target medical claim history; and
causing a first electronic communication to be displayed by a client device, wherein the first electronic communication comprises an initiation of an intervention to be performed.
